(19) United States
(12) Patent Application Publication
(10) Pub. No.: US 2016/0228117 A1
(43) Pub. Date: Aug. 11, 2016

(54) SUTURE LOCK

(71) Applicant: PETER SCOTT BORDEN, TORRANCE, CA (US)

(72) Inventor: PETER SCOTT BORDEN, TORRANCE, CA (US)

(21) Appl. No.: 14/968,808

(22) Filed: Dec. 14, 2015

Related U.S. Application Data
(60) Provisional application No. 62/092,177, filed on Dec. 15, 2014.

(51) Int. Cl. A61B 17/04

(52) U.S. Cl. CPC A61B 17/0401; A61B 2017/044; A61B 2017/0445; A61B 2017/0464

(57) ABSTRACT

A lock and method for suspending suture(s) from bone and providing a secure fixation of the suture(s) within the lock including a generally cylindrical shell having a threaded interior wall, a screw adapted to be driven into shell and a plug separately positioned within the shell below the screw or made unitary with and positioned at the driven end of the screw.

### CROSS REFERENCE TO RELATED APPLICATION

This application claims the benefit of priority of U.S. provisional patent application No. 62/092,177, filed Dec. 16, 2014, which is incorporated by reference herein.

### FIELD OF INVENTION

The invention relates generally to the field of surgery and is intended to allow for improved fixation of soft tissue or bone within a predrilled bone tunnel or socket.

### BACKGROUND

Devices and methods of suspending suture(s) from bone are known. However, these devices and methods typically require knotting the suture(s), and expose the suspended suture(s) to loosening and/or untying.

### SUMMARY

The suture lock and method of implementation according to the present invention overcome the drawbacks of known suture lock devices and methods of implementation by providing multiple points of fixation of the suture and without knotting the suture.

This invention relates to an apparatus and method for suspending suture from bone and providing a secure fixation of the suture within the device. The specific design of the device allows for tensioning of various sized suture material or suture tape that has been passed through the device. After the sutures are pulled through the apparatus member to the desired position of fixation, a second internal member of the apparatus is screwed into the apparatus which advances a third inner piece, a plug which provides for multiple points of fixation against the suture. An alternative design of this concept would involve combining the screw and third piece plug into one piece that accomplishes both tasks. The unique internal compression mechanism allows for three different points of compression against the suture material which allows for superior fixation. This accomplishes knotless fixation.

The present suture lock is intended to be used in the reconstruction of soft tissue disruption such as ligament or tendon tears. This lock and its implementation avoid the need for suture tying and are intended for use in various areas of the musculoskeletal system. By providing secure fixation of the tissue within the bone, the body part may be rehabilitated more aggressively and thereby reducing postoperative complications such as stiffness of a joint and improve recovery time following surgery.

Implementation of the present suture lock includes drilling or reaming a desired size hole into bone. Sutures are then passed through the hole and then passed through the lock. The lock is then inserted into the hole and pressed against the bone. The sutures are then pulled through the lock to the desired tension. The tissue intended for fixation is then attached to the opposite end of the sutures. Once satisfactory positioning of the tissue is achieved, the lock is operated or tightened by driving the screw inward until the suture is fully compressed. This allows for completion of the ligament fixation.

The present invention allows for fastening of suture through a predrilled hole by compression of the suture between pieces of the apparatus. Prior to passing the suture through the device, it is attached to tissue (such as ligament, tendon, or bone) that is intended to be “fixed”.

These and other embodiments, features, aspects, and advantages of the invention will become better understood with regard to the following description, appended claims and accompanying drawings.

### BRIEF DESCRIPTION OF THE DRAWINGS

The foregoing aspects and the attendant advantages of the present invention will become more readily appreciated by reference to the following detailed description, when taken in conjunction with the accompanying drawings, wherein:

FIG. 1 is a perspective view of a preferred embodiment suture lock socket and plug of a preferred suture lock of the present invention;

FIG. 2 is a perspective, cross-sectional view of the FIG. 1 embodiment;

FIG. 3 is a perspective, exploded, cross-section view of the socket, plug and screw of the FIG. 1 embodiment shown with two sutures positioned within the suture lock;

FIG. 3 is a side cross-section view of FIG. 3 with screw is a tightened, locked position.

FIGS. 5A-E are detailed views including a front view, side cross-sectional view; side cross-sectional view open for suture; perspective view taken from above at about a 45-degree angle; and side cross-sectional view closed or locked position; and,

FIGS. 6A-E are further detailed views including the front view with captured suture material, front view open for capturing suture material, side cross-sectional view open for suture; side cross-sectional view closed or locked and with captured suture material; and perspective view taken from above at about a 45-degree angle as shown in FIGS. 5A-E.

Reference symbols or names are used in the Figures to indicate certain components, aspects or features shown therein. Reference symbols common to more than one Figure indicate like components, aspects or features shown therein.

### DETAILED DESCRIPTION

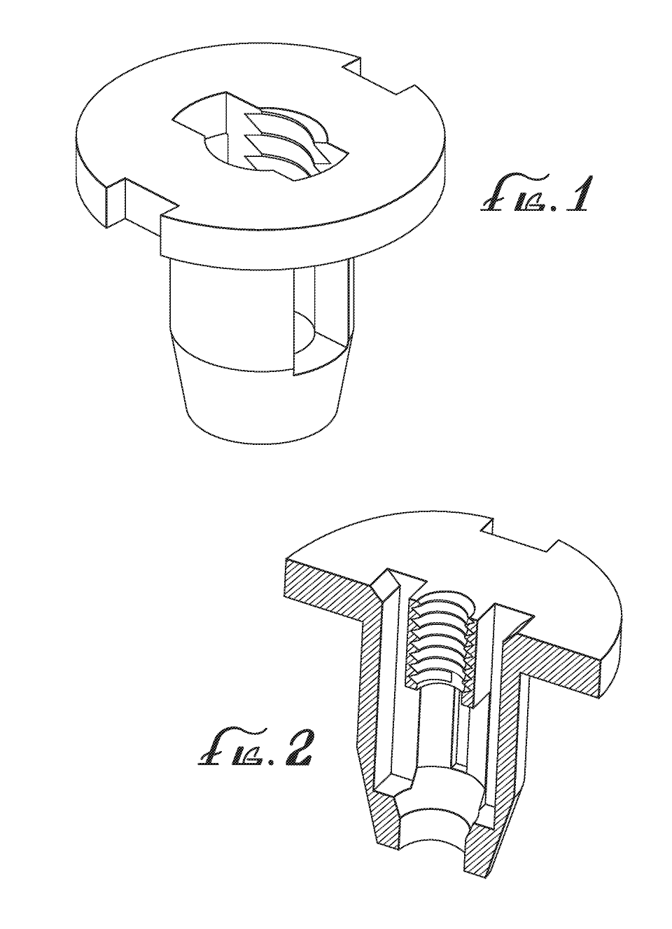

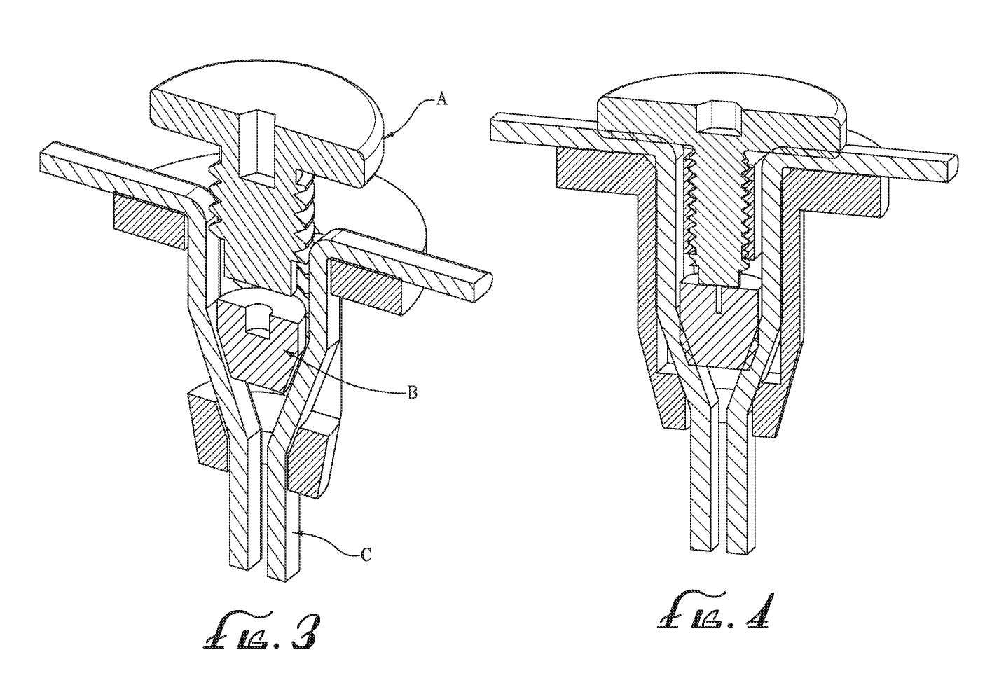

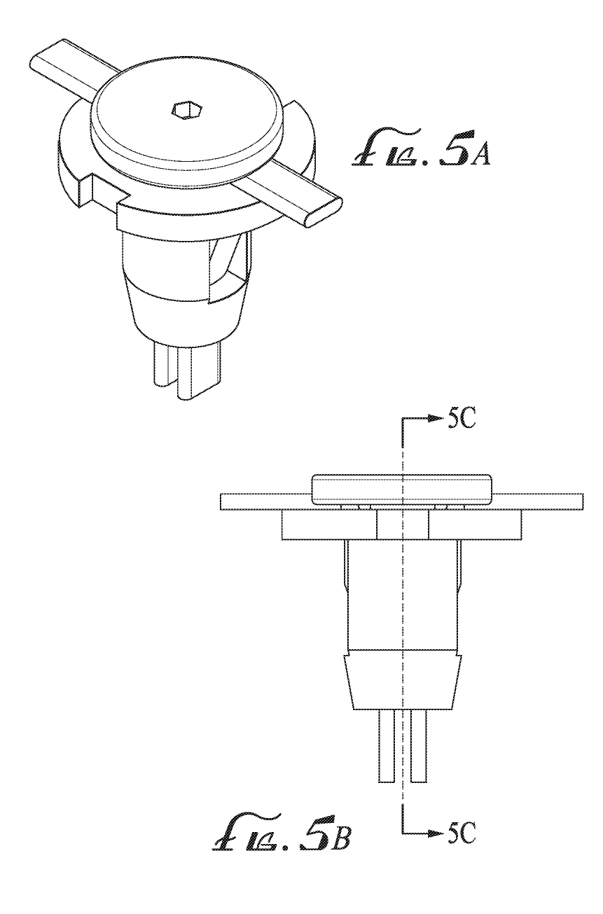

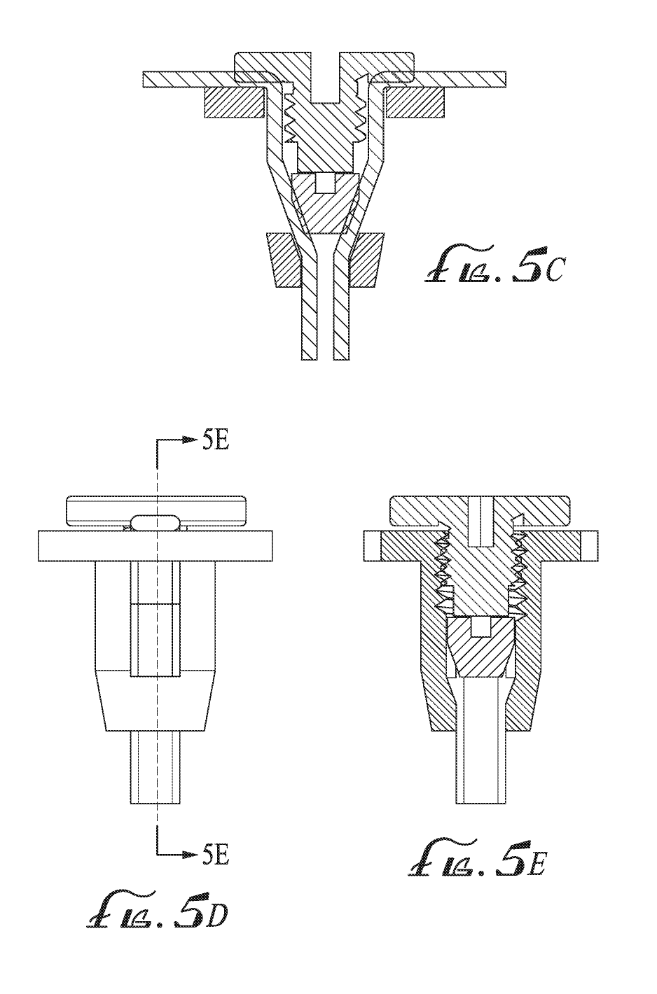

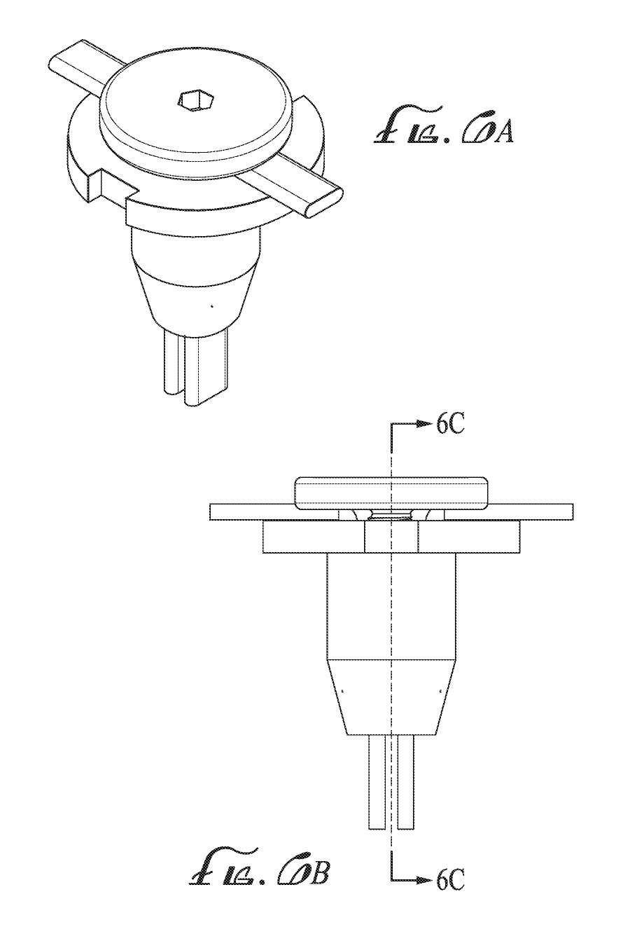

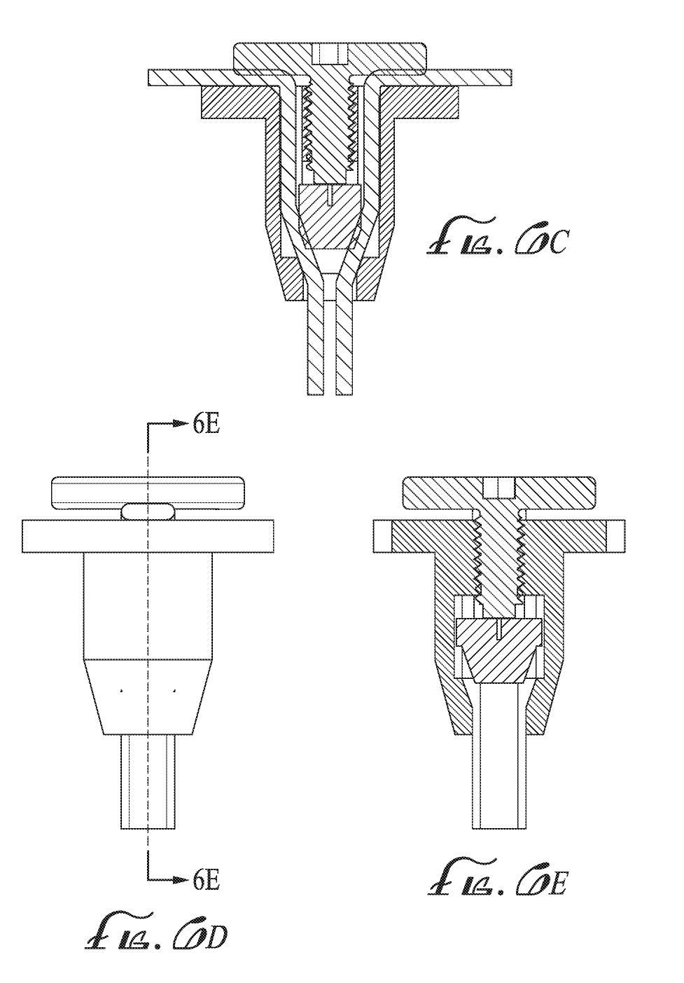

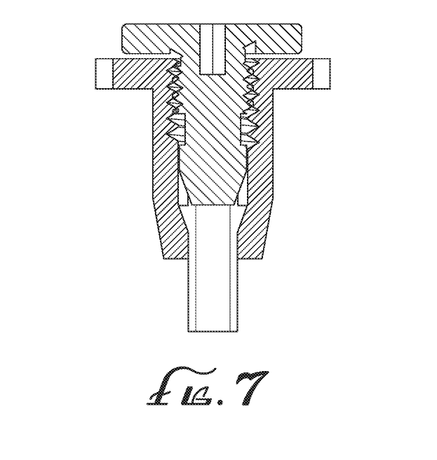

In accordance with embodiments described herein, the lock involves an outer piece hollow, shell, also referred to as a body that is inserted into a drilled hole within bone. The shell consists of a shaft and wider diameter collar or top flange, preferably of a one-piece construction (FIG. 1). Within the canal of the proximal end of the shaft lies a threaded component intended for an inner piece screw (FIG. 3). The inner piece (screw) (FIG. 3a) is advanced into the outer piece threaded construct by a screw driver. As the inner piece is advanced, it engages a second inner piece (compression plug) (FIG. 3b) within the outer piece (shell) that advances towards the tip of the outer piece (shell). The screw and the plug may be made as a unitary piece or component of the suture lock described herein. The apparatus may be made of various types of material to include: PEEK, composite, or metal.

The clinical application of the apparatus involves passing sutures (FIG. 3c) through the tip of the outer piece (shell) and up through the central canal of the outer piece (shell) and out the top of the outer piece (shell). The inner piece (screw) is then advanced which also advances the inner piece (compression plug), thus, compressing the sutures at the tip of the outer piece (shell) and against the inner walls of the outer piece (shell). Finally, when the screw is advanced completely, it's “head” engages the sutures against the outer rim of the outer piece (shell) collar providing a third point of compression/fixation (FIG. 4).

The presently preferred internal screw-in mechanism shown herein provides for multiple points of fixation of various sized (thickness and width) suture material. The screw allows for adjustability of the suture tension by having the ability to back up the screw and retighten it. This allows for optimal positioning of the ligament reconstruction. For example, if the apparatus is used on both sides of the knee for an ACL reconstruction, the tissue graft can be advanced into the femur or tibia and adjusted from either end of the graft. This may avoid a significant issue associated with bone tendon bone reconstructions commonly referred to as “tunnel mismatch.” The presently described fixation lock and method of implementation also permits performing these types of reconstructions without the need for fixation within the socket (such as interference screws). The collar portion of the body or shell also can function to plug the hole preventing leakage of important fluids involved in the ligament healing process. The presently described lock also functions to permit fixation of tissue through small incisions which in turn avoids further soft tissue damage associated with more invasive exposures.

The present suture lock may be developed with slight variations of the internal or outer components for the same multiple point fixation.

The present suture lock may be used in various areas of the body such as the knee, shoulder, ankle, and hip.

Although specific embodiments of the invention have been described, various modifications, alterations, alternative constructions, and equivalents are also encompassed within the scope of the invention.

The specification and drawings are, accordingly, to be regarded in an illustrative rather than a restrictive sense. It will, however, be evident that additions, subtractions, deletions, and other modifications and changes may be made thereunto without departing from the broader spirit and scope of the invention as set forth in the claims.

What is claimed is:

1. A suture lock comprising:
a shell having a generally cylindrical shell body, an interior wall including threads extending along a predetermined length, an open bottom end, a top end and top end flange extending radially outward from said top end;
a screw having a flat bottom end, a top and a threaded shaft adapted to be driven into the shell body; and,
a plug positioned within the shell body between the open bottom end of the shell body and the screw bottom end;
whereby suture material positioned between the plug and the shell may be compressed upon rotation of the screw.

2. The suture lock of claim 1, wherein the screw is adapted to be loosened or tightened to adjust the tension of the suture material.

3. The suture lock of claim 1, wherein the components are made of the same material such as metal, PEEK, or a composite material.

4. The suture lock of claim 1, wherein the screw is adapted to permit application of accurate torque and compression of suture material.

5. The suture lock of claim 1 wherein the screw and the plug are of a unitary construction.
